(19) United States
(12) Patent Application Publication
(10) Pub. No.: US 2010/0111872 A1
(43) Pub. Date: May 6, 2010

(54) System and method for modulating and optimizing immunotherapy

(75) Inventors: Peter Alan Bretscher, Saskatoon (CA); Duane Howard Hamilton, Washington, DC (US)

Correspondence Address: FASKEN MARTINEAU DUMOULIN LLP, 2900 - 550 Burrard Street, VANCOUVER, BC, V6C 0A3, CA

(73) Assignee: UNIVERSITY OF SASKATCHEWAN, Saskatoon (CA)

(21) Appl. No.: 12/289,861

(22) Filed: Nov. 6, 2008

(51) Int. Cl. G01N 33/53; A61K 49/00

(52) U.S. Cl. 424/9.2; 435/7.1

(57) ABSTRACT

A method for modulating a subject's type of immune response to a cancer treatment. The method comprises the steps of: (a) obtaining a sample from subject; (b) assessing the sample to detect IgG antibodies or IgG isotypes in the sample; (c) if IgG antibodies or IgG isotypes are detected, then identifying and quantifying their expression; (d) comparing the quantified IgG antibodies or IgG isotypes with a control to determine the changes, and characterize the immune response. The changes in expression of IgG antibodies or IgG isotypes are indicative of the type of immune response to the cancer treatment. Steps (a) through (f) are repeated for each of a series of spaced-apart time points. The series of characterized immune responses are assessed to determine the subject's responsiveness to the cancer treatment. The cancer treatments are then modulated to affect desired changes in expression of IgG antibodies or IgG isotypes.

### FIELD OF THE INVENTION

The present invention relates generally to the use of serological testing as a method for optimizing a chemotherapeutic immunotherapy. More particularly, the invention relates to a method of optimizing chemotherapeutic immunotherapy by correlating and modulating a subject's cellular and humoral immune response.

### BACKGROUND OF THE INVENTION

The mammalian immune system is a complex collection of mechanisms within an organism that protects it against disease by detecting, identifying, and killing pathogens and tumour cells. The primary function of the immune system is to protect an organism from infection through a series of defense mechanisms including physical barriers, innate immune responses, and adaptive immune responses.

The occurrence of cancer in a subject generally represents a basic failure of the immune system. To assess the failure, analysis and identification of differences in the immune response between subjects that resist tumour growth versus those that suffer tumour progression is needed.

The immune system has a variety of mechanisms to both contain and combat infection. The immune system recognizes tumours, and mounts immune responses against them. When a pathogen, for example a cancer cell, breaches the physical barriers of the immune system, the system generally responds with a rapid innate immune response which is non-specific. When a pathogen evades the first two immune response mechanisms, the immune system generally responds with an adaptive immune response. The adaptive immune response is a specific response to the invading pathogen, and is activated by the innate response. When an adaptive immune response is generated, the immune system has adapted based on its first exposure to the pathogen through an immunological memory. It is the immunological memory that enables the adaptive immune system to specifically attack a particular pathogen. Immunological memory may be artificially generated through the use of vaccines.

Vaccination against acute infectious diseases such as smallpox and polio has been very effective, due to the rapid and increased immune responses of vaccinated individuals to natural infections. However, in contrast, effective vaccination against intracellular pathogens that cause cancer, has been largely unsuccessful to date. Vaccination against diseases such as polio virus, smallpox, and tetanus enable a rapid immune response upon natural infection by the pathogen. However, in the case of cancer, clinical observations have indicated the outcome of infection depends more upon the type of immunity generated rather than the speed with which that immunity occurs. In these cases, vaccination ensures a rapid, but ineffective humoral response, which is generally not efficacious against such intracellular pathogens.

The normal progression of a subject's immune response to most antigens is generally an initial cell-mediated response that shifts toward a secondary humoral response (antibody response). It is very difficult to alter the normal progression of an immune response. In the treatment of intracellular pathogens, for example cancer, subjects who produce a strong, stable, and predominantly cell-mediated response against the pathogen often contain the disease. However, it has been observed that those subjects producing only humoral or a mixed cell-mediated/humoral response often suffer chronic or progressive disease. For generation of an effective immune response against intracellular pathogens, it is critical that the immune system generates an appropriate immune response in order to confer protective immunity to the subject upon natural subsequent infections.

When a pathogen is detected, the immune system employs a decision-making process that results in the differential expression of different classes of immunity under different circumstances. Studies have shown that animals immunized to produce an antibody to an antigen may no longer be induced to express cell-mediated immunity, in the form of delayed-type hypersensitivity (DTH) to this antigen following a regimen of immunization that induced DTH in naive animals. The immune response becomes ‘locked’ into a humoral mode. This type of response is known as immune deviation, and in this case, humoral immune deviation (Asherson et al., 1962, J. Immunology, 9: 205-217).

An immune response to a particular antigen can become locked into both a humoral or a cell-mediated state. Some antigens can induce cell-mediated immunity, for example in the form of DTH but can not induce the formation of antibody. These antigens are identified as being minimally foreign for example, cancer cells, cells of an organ from a donor minimally different from the graft recipient, and cells infected by slow growing intracellular parasites. Cells of these types of antigenic entities have predominantly self or self-like antigens on their surface and are expected to display few foreign sites and thus, are generally susceptible to a cell-mediated but not humoral attack. Similarly, antibody-mediated mechanisms of attack are generally effective against cells that are more than just minimally foreign. For example, one mechanism of antibody-dependent attack is mediated by the complement system.

There is a need to develop method to quantify the immune response generated and to correlate the quantified the immune response to the specific type of immune response generated.

Further, there is a need to develop a method for optimization of the immune response for a particular disease condition, and for specific subjects. Moreover, there is a need to develop a method for the optimization of treatment of cancer, caused by intracellular pathogens. There is a further need to detect the generation of an immune response, particularly the type of immune response, and optimize that response according to the disease/infection type.

### BRIEF SUMMARY OF THE INVENTION

Some embodiments herein provide a method for modulating a type of immune response to a cancer treatment within a subject, the method comprising the steps of obtaining a sample from the subject, and assessing said sample to detect at least one IgG antibody or IgG isotype in the sample. If at least one IgG antibody or IgG isotype is detected, identifying and quantifying the IgG antibody or IgG isotype expression and comparing the quantified IgG antibody or IgG isotype with a suitable control. Changes in expression therebetween are determined, and those changes are indicative of the type of immune response to the cancer treatment. The type of immune response is then characterized, and each of the above steps are repeated for each of a series of spaced-apart time points. The series of characterized immune responses are assessed to determine the subject's responsiveness to the cancer treatment, and the cancer treatment is modulated to affect a change in the expression of the quantified IgG antibody or IgG isotype. In a further embodiment, at least one of IgG1 and IgG2a antibody is detected, quantified, and compared to a suitable control. The type of immune response is then characterized, and each of the above steps are repeated for each of a series of spaced-apart time points. The series of characterized immune responses are assessed to determine the subject's responsiveness to the cancer treatment, and the cancer treatment is modulated to affect a change in the expression of the quantified IgG1 and IgG2a antibody.

A further exemplary embodiment provides a method of characterizing a type of immune response to a cancer treatment in a subject comprising the steps of obtaining a sample from the subject, and assessing the sample to detect at least one of IgG1 antibody and IgG2a antibody in the sample. Where at least one of IgG1 and IgG2a antibody is detected, the antibody expression is quantified and compared to determine differences in the magnitude the expression therebetween. The differences in the expression of the quantified antibody are indicative of the type of immune response to the cancer treatment. In a further embodiment a ratio of expression of the quantified IgG2a antibody to IgG1 antibody is determined, and the ratio of expression of the quantified IgG2a antibody to IgG1 antibody is indicative of the type of immune response to the cancer treatment.

Another exemplary embodiment provides methods of modulating the type of immune response to a cancer treatment in a subject comprising the steps of obtaining a sample from the subject, and assessing the sample to detect at least one of IgG1 antibody and IgG2a antibody. If at least one of IgG1 and IgG2a antibody is detected, quantifying the antibody expression, and quantifying the ratio of expression of the quantified IgG2a antibody to IgG1 antibody. Each of the above steps are repeated for each of a series of spaced-apart time points, and the differences in the ratio of expression of the sample are compared at each of the series of spaced-apart time points. The type of immune response is characterized and assessed to determine the subject's responsiveness to the cancer treatment. The cancer treatment may then be modulated to affect a change in the ratio of expression of the quantified IgG2a antibody to IgG1 antibody. In a further exemplary embodiment, the quantified ratio of expression of the sample is compared to a ratio of expression of a suitable control, and changes in the ratio of expressions therebetween are determined. Changes in the ratios of expression are indicative of the type of immune response to the cancer treatment. The type of immune response is then characterized and the above steps repeated for each of a series of spaced-apart time points. The series of characterized immune responses are assessed to determine the subject's responsiveness to the cancer treatment, and the cancer treatment may be modulated to affect a change in the ratio of expression of the quantified IgG2a antibody to IgG1 antibody.

A further exemplary embodiment provides a method for modulating a type of immune response to a cancer treatment within a subject comprising the steps of obtaining a sample from the subject, and assessing the sample to detect at least one of IgG1 antibody IgG2a antibody. If at least one of IgG1 antibody and IgG2a antibody is detected, quantifying the antibody expression and comparing the quantified IgG1 and IgG2a antibody to determine the difference therebetween and, characterizing the type of immune response. Each of the above steps is then repeated for each of a series of spaced-apart time points, and the series of characterized immune responses are assessed to determine the subject's responsiveness to the cancer treatment. The cancer treatment is then modulated to affect a change in the expression of the quantified IgG1 antibody and IgG2a antibody.

According to one exemplary embodiment, the present invention provides a method of quantifying a type of immune response to a cancer treatment within a subject comprising the steps of obtaining at least two samples from the subject, and harvesting cells from the samples. The harvested cells from one of the samples is exposed to an anti-IgG1 antibody, and the harvested from the second of the samples is exposed to an anti-IgG2a antibody. The cells are incubated, and then washed with a buffer solution. The expression of IgG1 and IgG2a antibody in each of the samples is then quantified, and the quantified antibody is indicative of the magnitude of the type of immune response to the cancer treatment.

### BRIEF DESCRIPTION OF THE DRAWINGS

Embodiments of the present invention will now be described, by way of example only, with reference to the attached Figures, wherein:

FIG. 1 is an flow chart showing the generation of a typical immune response;

FIG. 2A is a chart showing an immune response to P815 tumour cells;

FIG. 2B is a chart showing an immune response to P815 tumour cells;

FIG. 3A is a chart and a companion gel showing an immune response to P815 tumour cells;

FIG. 3B is a chart and a companion gel showing an immune response to P815 tumour cells;

FIG. 3C is a gel showing immune response to P815 tumour cells;

FIG. 3D is a chart and a companion gel showing an immune response to P815 tumour cells;

FIG. 3E is a chart and a companion gel showing an immune response to P815 tumour cells;

FIG. 3F is a chart and a companion gel showing an immune response to P815 tumour cells;

FIG. 4A is a chart showing an immune response to L5178Y tumour cells;

FIG. 4B is a chart showing an immune response to L5178Y tumour cells;

FIG. 5A is a chart and a companion gel showing an immune response to L5178Y tumour cells;

FIG. 5B is a series of charts and companion gels showing an immune response to L5178Y tumour cells;

FIG. 6A is chart and a companion western blot showing an immune response to P815 tumour cells;

FIG. 6B is chart and a companion western blot showing an immune response to P815 tumour cells;

FIG. 7A is a chart showing an immune response to P815 tumour cells; and

FIG. 7B is a series of gels showing a time-related immune response to P815 tumour cells.

### DETAILED DESCRIPTION OF THE INVENTION

The present invention discloses a method for characterizing, quantifying, modulating and optimizing a subject's immune response to a cancer treatment. The prevalence of different Immunoglobulin G (IgG) isotypes in anti-tumour IgG antibody reflects the type and magnitude of cytokines produced by cancer-specific, particularly tumour-specific T cells and in turn, reflects the type and magnitude of the immune response.

Most cancer treatments result in removing or killing dividing tumour cells. In addition, these treatments may have the ability to not only reduce and eliminate tumour cells, but also modulate the type of immune response toward a predominantly cell-mediated immune response. This modulation of the immune response is readily achievable when cancer treatment is tailored to a specific subject, thus optimizing the cancer treatment for that subject. Detecting, quantifying, and assessing specific IgG antibody and IgG isotype expression longitudinally over a period of time enables the characterization of the type and magnitude of the immune response to a cancer treatment within a subject.

The immune system's response to antigens typically progresses through an exclusive cell-mediated, Type 1 helper T cells (Th1), response period prior to the onset of a humoral, Type 2 helper T cells (Th2), response involving antibody production. Antibody production is generally associated with a decline in delayed-type hypersensitivity (DTH) which is a component of the cell-mediated immune response. An exclusive cell-mediated response, when followed by antibody production, is generally also followed by the appearance of CD4-Th2 cells which at least partially inhibits a cell-mediated response.

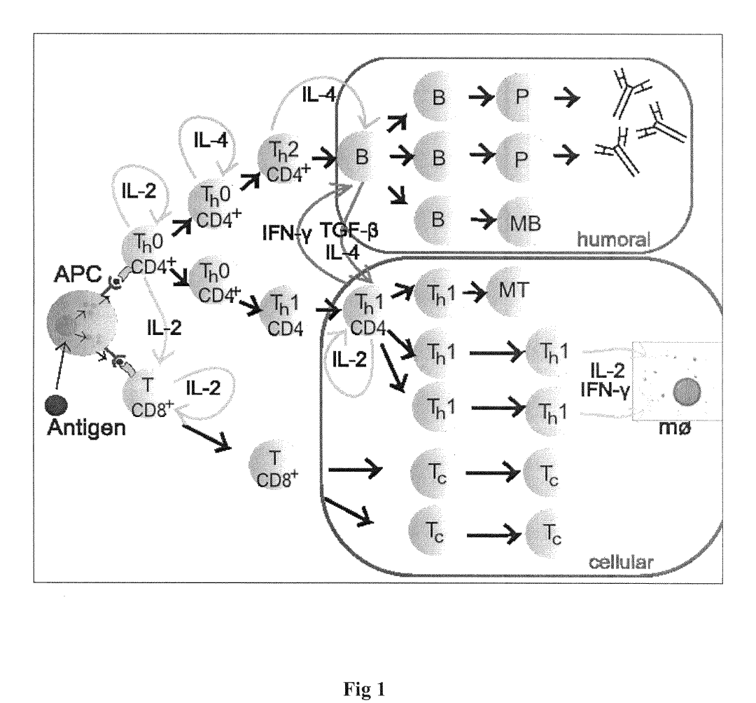

Mediating and monitoring the immune response to at least one antigen is a critical component in the development of a cancer treatment regimen. Characterizing and modulating the Th1/Th2 type of immune response within a subject enables optimization of the subject's cancer treatment by effecting a change in the type of immune response within the subject. FIG. 1 illustrates a typical immune response cascade showing the various components related to Th1 and Th2 immune responses.

Traditionally, an anti-tumour immune response in a subject was quantified using an ELISPOT assay and is reflective of the immune response occurring in that subject at a particular moment in time. This quantified immune response may show a large degree of variation between subjects, particularly subjects within the same experimental group. This variation is likely reflective of kinetic differences in the development of each subject. In order to overcome this variation, it has been discovered that quantifying the relative levels of subclasses of the IgG antibody may be used to indirectly assess the Th1/Th2 nature of the immune response.

IgG is the most abundant immunoglobulin found in the human body. It is typically equally distributed in both the blood and in tissue liquids constituting about 75% of serum immunoglobulins in humans. IgG molecules are synthesised and secreted by B cells. Immunoglobulin G (IgG) is a monomeric immunoglobulin of about 150 kDa. It comprises two identical heavy chains, γ, of about 50 kDa and two identical light chains of about 25 kDa. Each of the two heavy chains are linked to one another and to a light chain by disulphide bonds. The resulting IgG molecule is a tetramer having two identical halves which together form a Y-like shape. Each IgG has two antigen binding sites located at ends of the fork.

Typically, large amounts of pathogen-specific IgG1 antibodies correlate to the generation of IL-4 producing T-cells, and moreover with a largely Th2 immune response. Predominance of pathogen-specific IgG2a antibodies correlate to the generation of IFN-γ-producing T-cells and are generally associated with a predominantly Th1 immune response.

An effective immune response against a tumour antigen is generally associated with the expression of predominantly IgG2a antibodies, while progressive tumour growth is generally associated with the expression of predominantly IgG1 antibodies generated against the tumour antigens. The quantity of IgG1 and IgG2a antibody expression does not reflect the immune response at a specific moment in time, but is instead reflective of an integrated immune response generated over a period of weeks. This integrated immune response is generally more consistently detected among groups of subjects as compared to immune responses detected in those same subjects using an ELISPOT assay.

Continual detection, quantification, and comparison of IgG1 antibody and IgG2a antibody expression over a series of spaced-apart time-points enables characterization of the type and magnitude of the immune response to a cancer treatment within a subject. Assessment of the characterized immune response over the series of spaced-apart time-points enables the modulation of a subject's cancer treatment. This modulation of the cancer treatment facilitates the optimization of the type of immune response within a subject, particularly to a predominantly cell-mediated immune response which is indicative of an increased responsiveness of the subject to the cancer treatment.

Some exemplary embodiments of the present invention relate to a method for modulating and optimizing the type of immune response to a cancer treatment within a subject. The method generally comprises obtaining a sample from the subject and assessing said sample to detect IgG1 and IgG2a antibody in the sample. If IgG1 antibody is detected in the sample, then quantifying the antibody expression. If IgG2a antibody is detected in the sample, then quantifying the antibody expression. The quantified IgG1 and IgG2a antibody is then compared with a suitable control, and determining changes in the expression therebetween. Then, the type of immune response is characterized and each of the above steps are repeated for each of a series of spaced-apart time points. The characterized immune responses are assessed to determine the subject's responsiveness to the cancer treatment. A change in expression between the quantified antibody and the control are indicative of the type of immune response to the cancer treatment. The cancer treatment may be modulated to effect a change in the expression of IgG1 and IgG2a antibody expression. More specifically, where the immune response is shifting toward a Th2 or mixed Th1/Th2 response, the cancer treatment may be modified so as to modulate the immune response within the subject to a predominantly Th1 response.

A suitable control for the purposes of this invention may be for example, a clinically selected control, a stored dataset of results generated from studies of the presence and expression of IgG1 and IgG2a antibody in one or more population(s)of subjects responsive to cancer treatment, a stored dataset of results generated from studies of the presence and expression of IgG1 and IgG2a antibody in one or more population(s) of subjects non-responsive to cancer treatment, a stored dataset of results generated from studies of the presence and expression of IgG1 and IgG2a antibody in samples collected from a single subject responsive to cancer treatment over an extended period of time, a stored dataset of results generated from studies of the presence and expression of IgG1 and IgG2a antibody in samples collected from a single subject non-responsive to cancer treatment over an extended period of time, or combinations thereof. A clinically selected control may be for example, a physiological specimen responsive to a cancer treatment or a physiological specimen non-responsive to cancer treatment.

Cancer may be treated by surgery, chemotherapy, radiation therapy, immunotherapy, monoclonal antibody therapy, or other methods known in the art as well as combinations of treatments. The selection of a cancer treatment is often dependent upon the location and grade of the tumour and the stage of the disease, as well as the general state of the subject. Numerous experimental cancer treatments are also continually in development.

A sample for the purposes of this invention may be for example cells, tissues, and fluids, more particularly non-cancerous cells, cancerous cells, tumour cells, blood, plasma and combinations thereof.

Further exemplary embodiments of the present invention relate to detecting and quantifying, in a series of samples taken over a period of time, the expression of IgG1 and IgG21 antibody, generating a ratio of expression of IgG2a to IgG1 as disclosed in Example 4 herein, to modulate and optimize the type of immune response within a subject. In the event the ratio of expression is decreasing and shifting toward a predominantly Th2 or mixed Th1/Th2 response, the cancer treatment may be modified such that the immune response is modulated to a predominantly Th1 response and the ratio of expression increased.

Moreover, the immune response to a cancer treatment within a subject indicates a predominately Th1 response wherein the ratio of expression is greater than at least about 100; a mixed Th1/Th1 response wherein the ratio of expression is greater than about 1 and less than about 100, and a predominantly Th2 response wherein the ratio of expression is less than at least about 2. Furthermore, the immune response to a cancer treatment within a subject indicates a predominately Th1 response wherein the ratio of expression is greater than at least about 90, 80, 70, 60, 50, 40, 30, 20, 10, 5, and 4. The immune response to a cancer treatment within a subject indicates a predominantly Th2 response wherein the ratio of expression is less than at least about 2, 1.5, 1, 0.5, 0.4, 0.3, 0.2, 0.1, 0.01, 0.001, and 0.0001.

In a further exemplary embodiment, the immune response of a subject to a cancer treatment may be modulated and optimized by calculating the ratio of expression of IgG2a to IgG1 for a sample, and comparing the sample's ratio of expression to the quantified ratio of expression of a suitable control. A change in the ratio of expression of the sample compared to the ratio of expression of the control indicates the types of immune responses generated over time, and the clinical responsiveness of the subject to the cancer treatment.

The clinical responsiveness of a subject to a cancer treatment is typically expressed as positive or negative. We discovered that an increase in responsiveness of a subject to a cancer treatment may indicate for example, an increase in the expression of IgG2a antibody, a decrease in the expression of IgG1 antibody, and combinations thereof. It may further indicate an increase in the expression of IgG2a antibody compared to a suitable control, a decrease in the expression of IgG1 antibody compared to a suitable control, an increase in the ratio of expression of IgG2a antibody to IgG1 antibody compared to the ratio of expression of a suitable control, and combinations thereof. A decrease in responsiveness of a subject to a cancer treatment may indicate for example, a decrease in the expression of IgG2a antibody, an increase in the expression of IgG1 antibody, and combinations thereof. It may further indicate, a decrease in the expression of IgG2a antibody compared to a suitable control, an increase in the expression of IgG1 antibody compared to a suitable control, a decrease in the ratio of expression of IgG2a to IgG1 compared to the ratio of expression of a suitable control, and combinations thereof.

Some exemplary embodiments of the present invention relate to methods for characterizing the type of immune response to a cancer treatment within a subject. A sample is obtained from a subject and, the sample is assessed to detect IgG1 and IgG2a antibody. If IgG1 and IgG2a antibody is detected in the sample, then quantifying the antibody. The magnitude of quantified IgG1 and IgG2a antibody is compared, and differences in the magnitude the expression determined. The differences in the magnitude of expression of the quantified antibody are indicative of the type of immune response to the cancer treatment.

In a further embodiment, the ratio of expression of the quantified IgG2a antibody to IgG1 antibody may be quantified. The ratio of expression of the quantified IgG2a to IgG1 antibody is indicative of the type of immune response to the cancer treatment.

Quantification of the two subclasses of antibody, IgG2a and IgG1, may be conducted using an enzyme immunoassay. This assay involves obtaining at least two samples from a subject, harvesting the cells from the samples, exposing the cells from one sample to an anti-IgG1 antibody and the second sample to an anti-IgG2a antibody, incubating the cells from the samples, washing the cells from the samples with a buffer solution, and quantifying the expression of IgG1 and IgG2a antibody in the first and second samples. The quantified antibody indicates magnitude of type of immune response generated.

A major mechanism by which a pathogenic disease, particularly a tumour, evades the generation of an immune response in a subject, is the deviation from an effective Th1, cytotoxic T lymphocyte response to a less effective response having a Th2 component. As shown in the Examples below, following primary murine tumour implantation, resistance of tumour progression correlates with a Th1 response and IgG2a antibody production. Tumour progression correlates with a mixed Th1/Th2 response and the production of IgG1 and IgG2a antibodies or an exclusive Th2 response.

### EXAMPLES

The following Examples illustrate methods of the present invention for optimizing chemotherapeutic immunotherapies. It is to be understood these examples should not be considered as limitations, and that the experimental data are for illustration only.

### Cell Lines, Immunoassay, Western Blotting and Animals

### Animals

DBA/2J mice were obtained from Jackson Laboratories (Bar Harbor, Me., USA) and from the University of Saskatchewan animal colony. Mice were maintained in, pathogen-free conditions and were routinely screened to ensure that they were free of sub-clinical viral and bacterial infections. Mice employed within each experiment were of the same sex and typically between the ages of 6-12 weeks. All experiments were performed under the strict guidelines of the Canadian Council on Animal Care.

### Tumour Cell Lines

The P815 cell line (TIB-64, Lot#2310374) was obtained from the American Type Culture Collection, Rockville, Md., USA. The L5178Y cell line is a thymoma induced by methylcholanthrene in ether, provided by Dr. R. J. North, Trudeau Institute, Saranac Lake, N.Y., USA. Tumour cells (0.02 ml) were implanted intradermally or subcutaneously.

### ELISPOT Assay

The ELISPOT assay was used for enumerating single antigen-specific cytokine producing cells due to its sensitivity and the demonstrable antigen-specificity of the cells it detects. ELISPOT was employed for the detection of antigen specific IFNγ-producing T cells. This was performed on spleen cells plated directly ex vivo into ELISPOT trays without an in vitro pre-culture step. The assay reflects the in vivo activity of the cells. High cell densities of at least 10⁶ cells/well are required for optimal detection of spot-forming cells, and only at a high density of cells is the number of spots detected linearly related to the number of primed cells plated. The number of spots is linearly proportional to the number of primed cells plated, even if these are well below a million cells/well. The formation of IFNγ spots is antigen-dependent and abrogated by depleting the antigen-primed cells of T cells.

96-well nitrocellulose-bottomed plates (Poly-filtronics, Rockland, Mass.) were coated with purified anti-IFNγ antibody R4-6A2 (Pharmingen, San Diego, Calif.) by incubating the plates overnight at 4° C. with 100 μl/well of antibody at 1.25 μg/ml in 50 mM carbonate buffer, pH 9.6. Spots appeared to be of highest quality when the coating buffer was between pH 9.0 and 10. The plates were emptied and rinsed once with 200 μl of complete RPMI medium. To reduce non-specific binding of proteins at later stages in the assay, the plates were incubated for at least one and up to three hours prior to addition of spleen cells with an additional 200 μl of complete RPMI.

The cells from the mice were added to the wells at densities ranging from 1.25×10⁵ to 1×10⁶ cells per well. The antigen was added to the wells. The assay was performed in triplicate wells with antigen stimulation for each cell density. The seeded plates were placed undisturbed in a 37° C. incubator for 8 hours. After incubation, the plates were rinsed once with distilled water and washed thoroughly with phosphate buffered saline (PBS) containing 0.05% Tween 20 (PBST). Biotinylated anti-IFN-γ antibody, XMG1.2 (Pharmingen) at 1.25 μg/ml in PBST was added to plates at 100 μl/well and incubated at 4° C. overnight. The plates were washed again with PBST and 100 μl of alkaline-phosphatase streptavidin (Jackson Immunoresearch Laboratories, West Grove, Pa.) at a concentration of 0.2 μg/ml in PBST was added to each well and incubated at room temperature for 1.5 hours. The plates were washed by repeated submersion in distilled deionized water (ddH₂O). Spots were developed by the addition of 100 μl/well of NBT/BCIP substrate (Boehringer-Mannheim, Germany) in substrate buffer prepared according to the manufacturer's instructions. After spot development, the plates were emptied, rinsed thoroughly with ddH₂O and allowed to dry. Spots were counted with the aid of a dissecting microscope.

### Western Blotting

Tumour cells grown in vitro were re-suspended at 10⁸ cells/ml for P815 and 3×10⁷ cells/ml for L5178Y in PBS. Samples were freeze-thawed, lightly sonicated and diluted 1:1 with Laemmli sample buffer (Bio-Rad Laboratories, CA, USA) and boiled for 4 minutes. A 200 μL volume of sample was run on a 4% stacking and 10% separating sodium dodecyl sulphate polyacrylamide gel. The separated proteins were blotted onto a 0.45 μm nitrocellulose membrane (Bio-Rad Laboratories) using a trans-blot semi-dry transfer cell (Bio-Rad). The membrane was blocked for 1 to 2 hours at room temperature on a rocker with a 5% blotting grade non-fat dry milk blocker in PBST (or PBS when using fluorescent secondary antibodies). After blocking, the membrane was assembled into a mini protean II multiscreen apparatus (Bio-Rad). Mouse serum was diluted in a 5% milk in PBST solution and 400 μL of the sample dilution was run in each lane. The membrane was incubated overnight at 4° C. on a rocker. The membrane was then washed and incubated with horseradish peroxidase-conjugated (HRP) goat anti-mouse IgG1 or IgG2a (Southern Biotechnology Associates Inc., AL, USA) or IRDye 800™ conjugated goat anti-mouse IgG1 (Rockland Immunochemicals, Gilbertsville, Pa., USA) and Alexa-Fluor® 680 conjugated goat anti-mouse IgG2a (Invitrogen) antibodies at a dilution of 1:5,000 in our 5% milk in PBST blocking solution for 1 hour at room temperature. The membrane was developed using Immun-Star™ HRP (Bio-Rad Laboratories) chemiluminescence kit as per the manufacturer's instructions. When utilizing fluorescent secondary antibodies, bands were visualized using the Licor Odyssey Imaging System (Licor, Lincoin, Nebr., USA).

### Example 1

### Development of Diagnostic Enzyme-Linked Immunoassay (ELA)

Traditional measurement of anti-tumour responses are detected using an ELISPOT assay. These measurements are reflective of the immune response occurring at a particular moment point in time and often show a large degree of variation between animals. An antibody-dependent assay was developed in order to measure relative amounts of antibody that reflect an integrated immune response over a period of time, and to facilitate the assessment of changes in the immune response in individual animals over time. The ELA antibody assay enables measurement of antibodies over time using the principle that antibodies have a half-life in circulation measured over a period of several days. Further, the assay enables an indirect measure of the magnitude of the anti-tumour immune response. Data collected from samples taken from a subject represent about the average immunity expressed over the previous week

This assay is used to quantify the relative predominance tumour-specific IgG1 and IgG2a antibodies. This assay demonstrates the correlation between a predominance of IgG2a antibodies with tumour regression, and between a mixed IgG1/IgG2a or a predominant IgG1 antibody response to tumour antigens and tumour progression. The ratio of expression of tumour-specific IgG2a to IgG1 antibodies can change over time. This ratio correlates with the rate of tumour growth. Mice with spontaneously regressing tumours maintain a relatively high ratio of tumour-specific IgG2a:IgG1 antibodies, while in mice with progressively growing tumours, this ratio may be initially be high, but subsequently decreases as tumour growth progresses.

L5178Y cells were harvested from in vitro culture, washed 4 times in sterile PBS and re-suspended at 3.3×10⁷ cells/ml. The cell suspension was freeze/thawed, and sonicated for 24 cycles on ice using a Branson Sonifer 450 (Branson, Danbury, Conn., USA) set at 50% duty cycle. When required, the soluble and insoluble fractions were separated by spinning the resulting suspension for 30 minutes at a 20,000 relative centrifugal force (RCF). The soluble fraction was collected, and the insoluble fraction was re-suspended in an equivalent volume of fresh PBS (sonicated 3-4 times to re-suspend the pellet). The L5178Y antigen was stored at −20° C. until use.

96-well high protein binding polystyrene Immuno-Maxisorp Plates (NUNC, Denmark) were coated overnight at 4° C. with an equivalent of 3.33×10⁶ freeze-thawed L5178Y cells per well in PBS. Plates were washed twice with 200 mL PBST per well, and blocked for 2 hours at 37° C. with 200 mL of PBS supplemented with 10% heat inactivated calf serum (Gibco Laboratories, Grand Island, N.Y., USA). Plates were washed twice with 200 mL PBST per well. Serum samples were diluted in 1/50 in PBS supplemented with 10% calf serum, and two-fold serial dilutions of the sera were performed in a 96-well low protein binding polyethylene plates (NUNC). Sera dilutions were transferred to the Immuno-Maxisorp test plate, and were incubated for 2 hours at 37° C. Test plates were washed six times with PBST, and 100 mL of horseradish peroxidase-conjugated goat anti-mouse IgG1 or IgG2a (Southern Biotechnology Associates Inc., AL, USA) were added in each well of the test plate at a dilution of 1:5000 in PBS supplemented with 10% calf sera. The plate was incubated at 37° C. for one hour then washed 6 times with PBST. 100 mL ATBS (2,2′-azino-di(3-ethyl-benzthiazoline-6-sulfonate)) and 1-component microwell peroxidase substrate (Kirkegaard & Perry Laboratories, Inc, Gaithersburg, Md., USA) were added to each well. The test plate was incubated for 20 minutes at room temperature in the dark. The test plate was read using an E-max microplate reader (Molecular Devices Corporation, Sunnyvale, Calif., USA) at a wavelength of 405 nm, and resulting data were collected and analyzed using the Softmax Professional version 2.2.2 (Molecular Devices Corporation) computer program. The signal from serum from naive mice was never above the values obtained from the conjugate blank control wells, an adsorbance of 0.065. The serum titer was assigned using an adsorbance cut-off value of 56.

### Example 2

### Nature of Immune Response in P815 and L5178 Tumour Models

A challenge model that did not cause progressive tumour growth was identified for each tumour model. This challenge was expected to make the mice resistant to a normally lethal challenge. A comparison of the Th1/Th2 nature of the immune response generated in mice challenged with a tumour dose was conducted. Mice were made resistant to P815 by excision or “low dose” subcutaneous priming in the footpad. Mice were made resistant to the L5178Y lymphoma by intradermal “low dose” priming in the belly. Critically, progressive tumour growth in naive mice is associated with a mixed Th1/Th2 or predominant Th2 response, and mice made resistant to the same challenge generated a predominant Th1 response in both the P815 and the L5178Y systems.

### P815 Mastocytoma Tumour Model

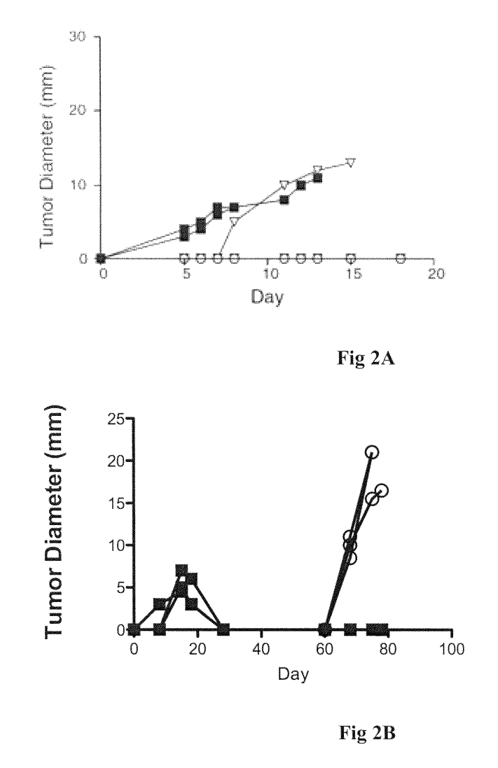

Eight mice were challenged with an injection of 10⁵ P815 cells subcutaneously into their right hind footpad. After a period of 15 days, these mice as well as two naive mice were implanted with 10⁵ P815 cells intradermally on the belly. Tumour progress was assessed after the challenge shown in FIG. 2a where ▪ represents mice receiving 10⁵ P815 implanted intradermally on Day 0; Δ represents mice receiving 10⁵ P815 subcutaneously injected into the footpad on Day −15 and 10⁵ P815 implanted intradermally on Day 0; and ∘ represents mice receiving 10⁵ P815 subcutaneously injected into the footpad on Day −15. Seven of the eight mice that received the challenge did not suffer progressive tumour growth. Moreover, about 70% of mice challenged with the initial 10⁵ P815 cells resisted a subsequent, normally lethal challenge of 2×10⁶ P815 cells implanted intradermally into their belly, when given a minimum of 2 weeks rest following the challenge. This challenge strategy was performed on more than 100 mice, and consistently rendered at least about 70% of the mice resistant to a normally lethal challenge of the P815 tumour.

A second group of mice eight were challenged in the footpad with an injection of 10⁵ P815 cells as described above. These mice and aged-matched naive control mice were given a normally progressive tumour challenge of 2×10⁶ P815 1-month following the initial challenge. The mice were sacrificed 10-days post challenge. Spleen cells from the sacrificed mice were enumerated using the ELISPOT assay, disclosed above, for P815-specific IFN-γ- and IL-4-producing cells.

### L5178 Lymphoma Tumour Model

Three mice were challenged with 10³ cells implanted intradermally as disclosed above. Tumour growth was assessed after receiving the challenge. Each of these mice spontaneously resolved their tumours by about Day 25 as shown in FIG. 2b where ▪ represents mice receiving 10³ L5178Y implanted intradermally on Day 0 and 10⁶ L5178Y on Day 60; and ∘ represents mice receiving 10⁶ L5178Y implanted intradermally on Day 60. After a period of 60 days, these mice as well as three age-matched naive mice, were challenged with 10⁶ L5178Y cells implanted intradermally on the belly. This dose of 10⁶ cells is normally a lethal tumour challenge. All mice that received the challenge did not suffer progressive tumour growth.

A second group of mice six were challenged with an intradermal implantation of cells 10⁶ cells in the belly. The mice were sacrificed 30-days post implantation by which time it was apparent which of the mice were rejecting their tumour and which were suffering from progressive tumour growth. The immune response generated against the L5178Y tumour was assessed in spleen cells from the sacrificed mice using the ELISPOT assay disclosed above. A significant predominance of tumour-specific IL-4-producing cells was evident in the spleen cells of mice suffering from progressive tumour growth indicating a predominatly Th2 immune response. IFN-γ-producing cells which correlate to a Th1 immune response were predominant in mice that had successfully contained the tumour.

For each tumour model, the ratio of the number of IFN-γ to IL-4 producing cells was analyzed. For each of the mice, a very clear, non-overlapping difference in the type of immunity expressed associated with regressing or progressing tumours was demonstrated. Progressive tumour growth was found where a mixed IFN-γ/IL-4 response was detected. Mice that rendered resistance to tumour growth exhibited a predominant IFN-γ response to the tumour antigens.

The above observations illustrate that resistance to tumour growth may be achieved by “low-dose” priming, and that tumour regression is associated with a predominant Th1 response to a tumour challenge. Effective “low dose” priming facilitates the establishing of “a Th1 imprint” upon the immune system.

### Example 3

### Analysis of the Production of Tumour-Specific Antibody in P815 and L5178 Tumour Models

Western blots were used to assess the production and relative predominance of tumour-specific IgG1 and IgG2a antibodies present in the sera of tumour-challenged mice. Specifically, the relative abundance of P815-specific IgG1 and IgG2a antibodies in the sera of individual mice given a normally non-progressive challenge of the P815 tumour was assessed. This challenge generally renders most mice resistant to a subsequent normally lethal challenge.

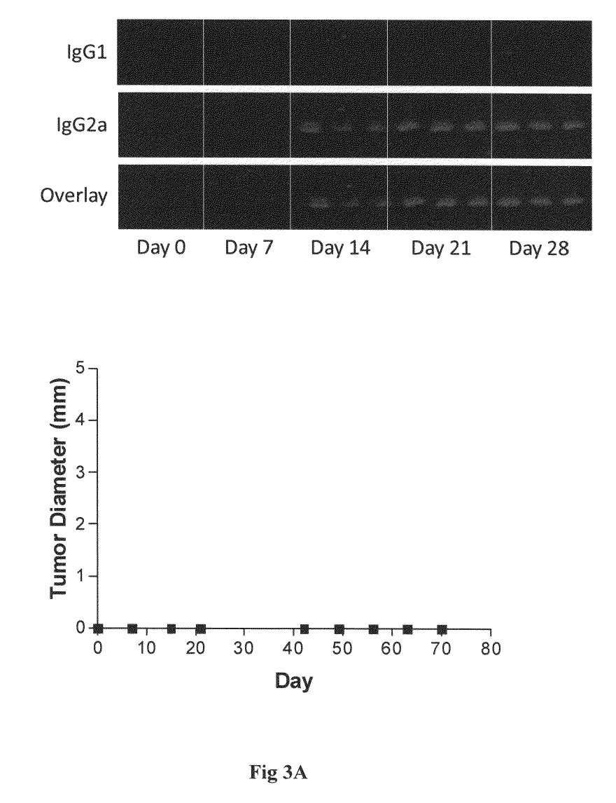

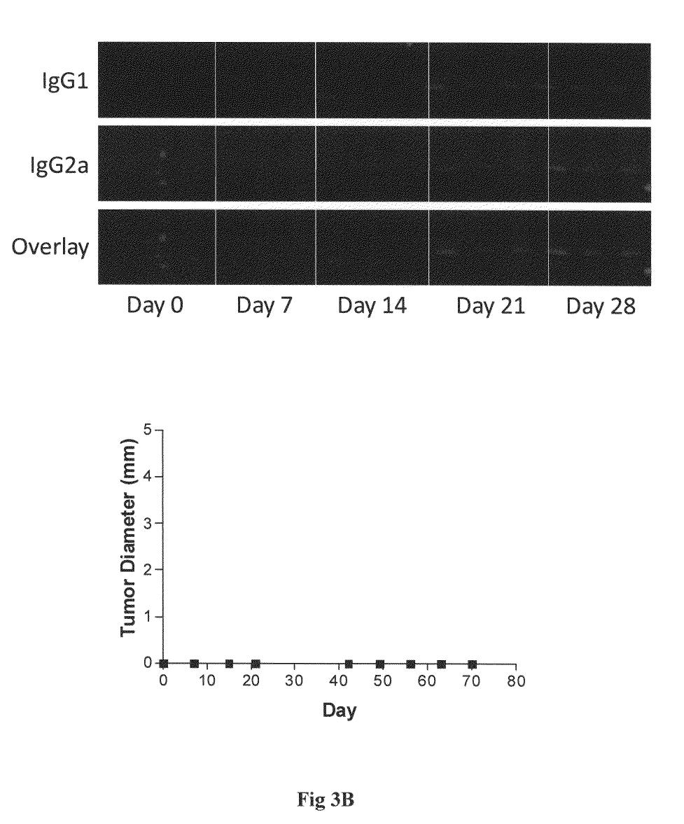

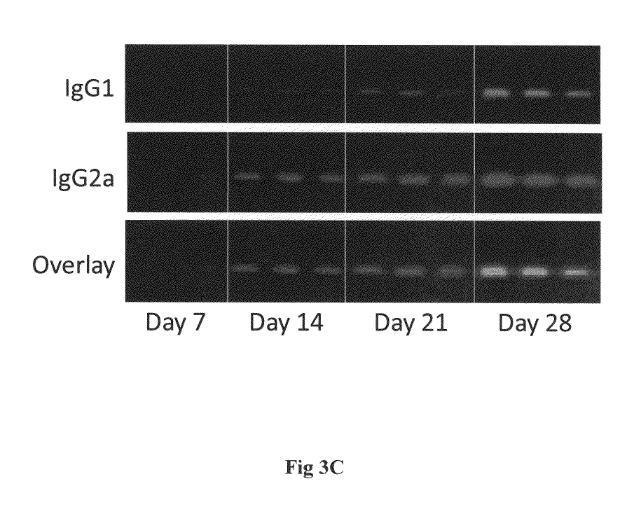

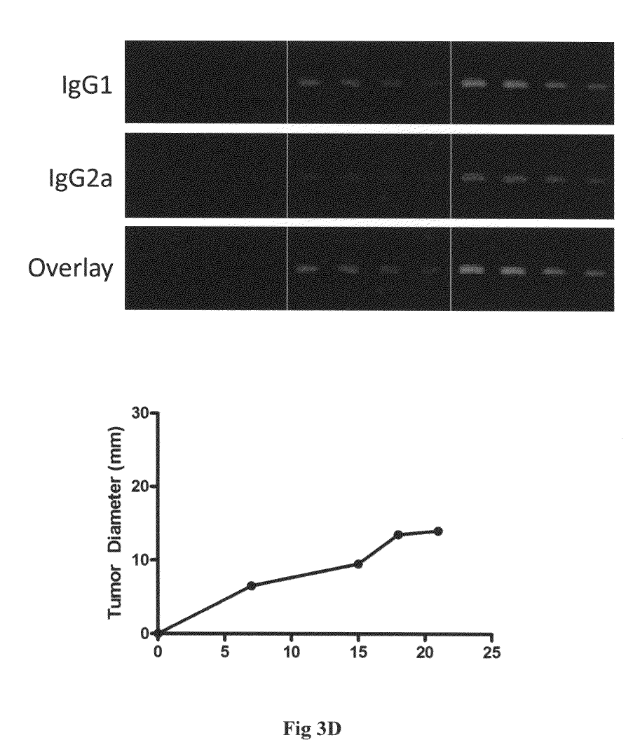

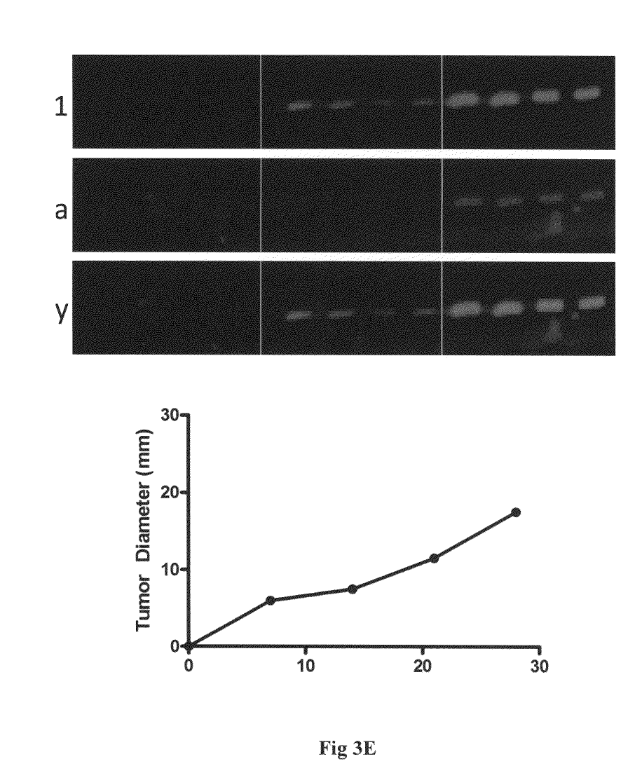

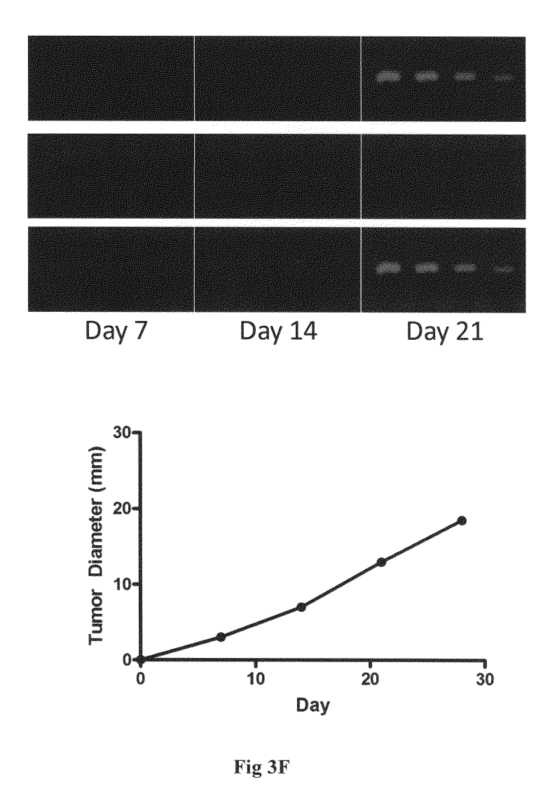

The mice were implanted with 10⁵ P815 cells subcutaneously into their hind footpad on day 0. The mice were serially bled over a course of 28 days, and the relative abundance of P815-specific IgG1 and IgG2a antibodies were assessed by Western blot. Three different types of immune response were represented in the various mice. The first type of immune response demonstrated is shown in FIGS. 3A and 3B. Day 0 in FIGS. 3A and 3B represents the day of initial challenge. FIGS. 3A and 3B illustrate the quantity of IgG1 and IgG2a in the sera of mice which subsequently resisted a normally lethal challenge of the P815 tumour 35 days following the initial challenge. FIG. 3A shows a predominance of P815-specific IgG2a antibodies, and little or no detectable P815-specific IgG1 antibodies. FIG. 3B, shows no detectable P815 antibodies which reflects a very predominant Th1 response.

The second type of immune response is illustrated in FIG. 3C. Mice given a normally non-progressive dose of 10⁵ P815 suffered progressive tumour growth. In this case, a predominance of tumour-specific IgG1 antibodies were detected in the mice serum. The IgG isotype pattern of FIG. 3C is very similar to that seen in mice given a standard lethal intradermal challenge on the belly as shown in FIGS. 3D-3F.

The third type of immune response is illustrated in FIGS. 3D, 3E and 3F which show the quantity of antibodies in the sera of naive mice implanted with a lethal dose of 10⁶ P815 cells intradermally on their belly. A predominance of tumour-specific IgG1 antibodies were detected in the mice serum.

These observations correlate with the data in Example 2 which discloses the relative number of IFN-γ- and IL-4-producing cells assessed using an ELISPOT assay. Using this Western Blotting methodology, it is shown that a priming strategy which rendered mice resistant to a normally lethal tumour challenge may be used to induce a stable Th1 response to tumour antigens.

### Example 4

### Immune Response Correlation with Variations in Tumour Progression/Regression

In order to assess why some mice suffer a more protracted course of tumour growth, Western blotting and ELA protocols were employed to indirectly assess the Th1/Th2 nature of the immune response generated over a period of time. The immune response was correlated with the corresponding rates of tumour growth. The ELA antibody assay enables measurement of antibodies over time and the Th1/Th2 nature of the anti-tumour immune response is inferred from the nature of the antibodies detected and measured at any particular time, as the sample collected from a subject and quantified represent about the average immunity expressed over the previous week. In both tumour systems (P815 and L5178Y), mice that were given a challenge that grew progressively generally had a higher ratio of IgG2a to IgG1 antibody when the antibody first was detected. This ratio decreased as the tumour growth progressed. A comparison of antibody responses in mice with rapidly growing tumours and in mice having a slower net or flat tumour growth, illustrates the differences in the types of anti-tumour immune responses generated. Tumour growth and the pattern by which the Th1/Th2 type of the anti-tumour immune response changes, is inferred from the relative predominance of IgG isotypes in the anti-tumour antibody found at different times post tumour implantation as is exemplified in the following Example.

### L5178Y

The relative abundance of L5178Y-specific IgG1 and IgG2a antibodies was assessed in mice having either progressive or spontaneously regressing L5178Y tumours.

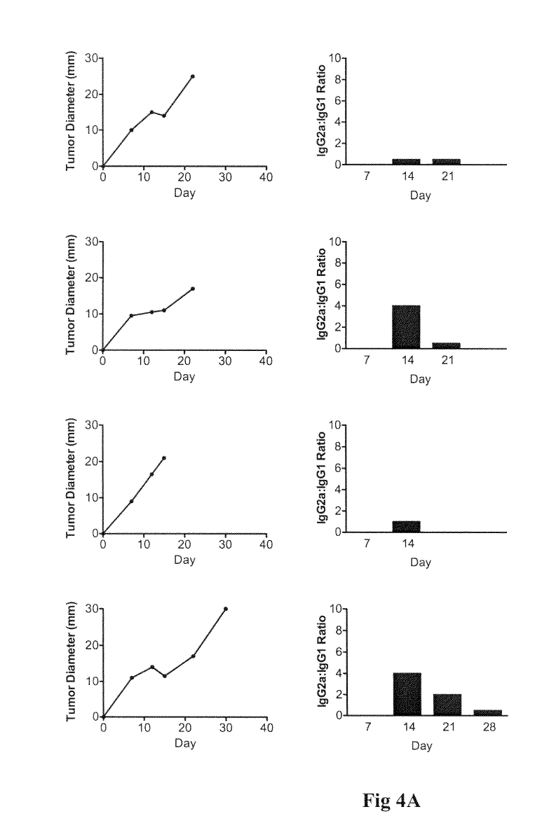

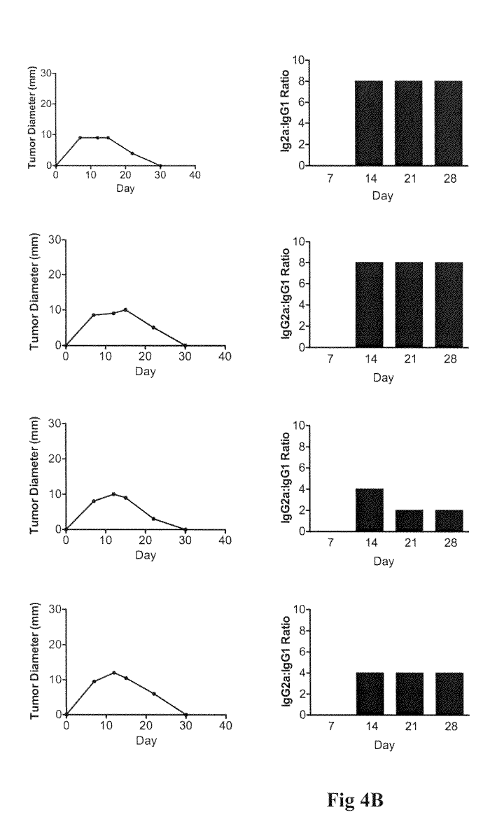

Twenty mice were implanted with 2×10⁶ L5178Y cells injected intradermally on day 0. Four mice suffered progressive tumour growth as is illustrated in FIG. 4B, shown in panels B-1, B-2, B-3, and B-4. Sixteen mice were resistant to tumour growth, results in four mice of these resistant mice as illustrated in, FIG. 4A, panels A-1, A-2, A-3, and A-4. The mice suffering progressive tumour growth were serially bled and the ratio of L5178Y-specific of IgG2a to IgG1 antibodies were assessed by ELA. In each case, the ratio was near zero illustrating a predominantly Th2 immune response.

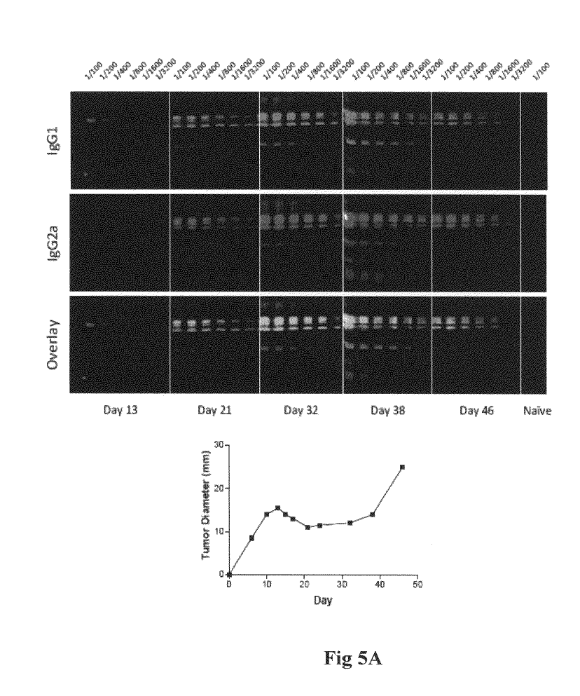

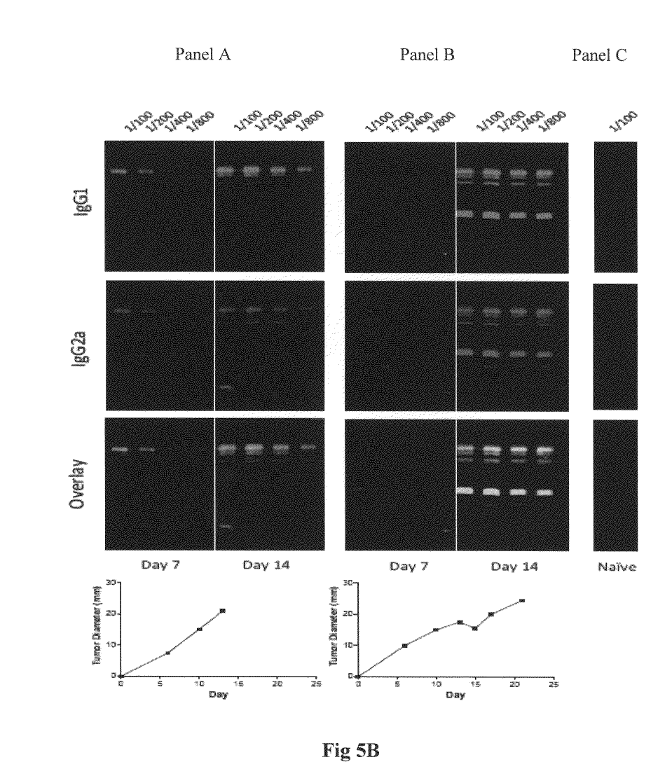

In a second experiment, mice were implanted 10⁶ L5178Y cells intradermally on day 0. Differing rates of tumour growth were observed, and the relative abundance of L5178Y-specific IgG1 and IgG2a antibodies were assessed by Western blot over the course of tumour progression. FIG. 5 illustrates the results in three mice. FIG. 5B, Panel A illustrates the expression of the antibodies in a mouse having rapid tumour growth; FIG. 5B, Panel B illustrates the expression of the antibodies in a mouse having moderate tumour growth; and FIG. 5A illustrates the expression of the antibodies in a mouse having slowed and flat tumour growth. In this case, there is a considerable period of time when IgG2a antibodies are predominant, reflecting a period when a predominant Th1 response occurs and when it is unclear whether tumour growth will progress. FIG. 5B, Panel C illustrates the antibody expression in the sera from naive mice have no detectable L5178Y-reactive antibodies in their sera. As is illustrated in FIG. 5, the predominance of IgG1 and IgG2a antibodies in each of three mice. As tumour growth slows, the ratio of expression of IgG2a to IgG1 antibodies increases.

### P815

Mice were implanted with 10⁵ P815 intradermally on their abdomen on day 0. The rate of tumour growth was assessed as was the relative abundance of tumour-specific IgG1 and IgG2a antibodies by Western blot.

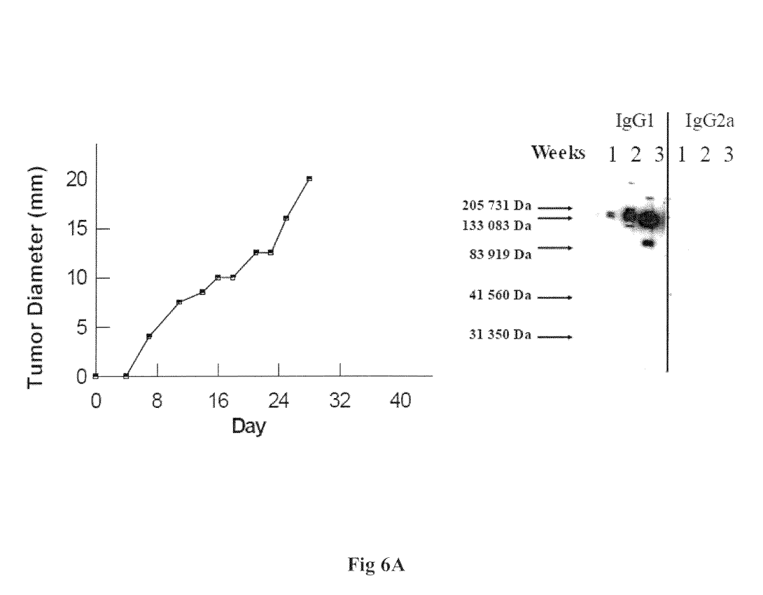

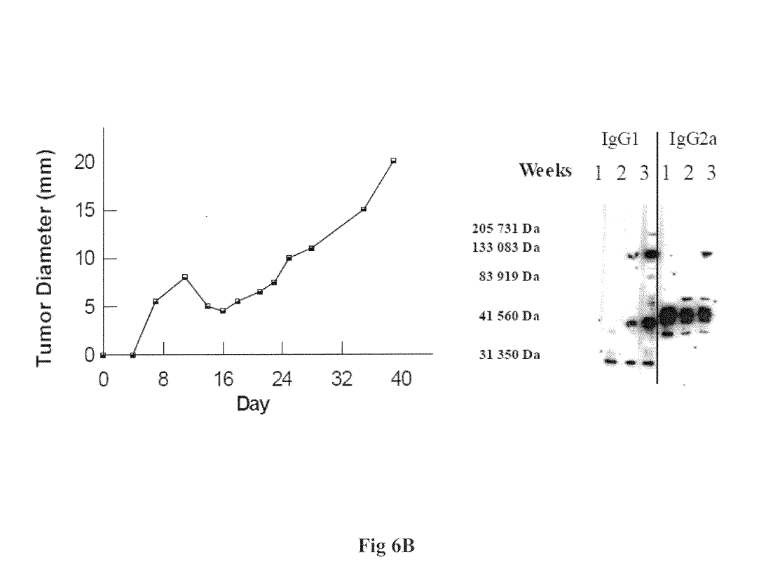

The pattern of tumour regression and progression in two of the mice is illustrated in FIG. 6. FIG. 6A illustrates a predominance of IgG2a antibody and slower tumour progression whereas, FIG. 6B illustrates a predominance of IgG1 antibody and in turn, progressive tumour growth.

### Example 5

### Correlation of Tumour Regression and Re-Emergence with Antibody Expression

The relative abundance of P815-specific IgG1 and IgG2a antibodies was assessed in a mouse exhibiting spontaneous tumour regression and subsequent tumour reappearance. The pattern of spontaneous tumour regression and subsequent tumour re-emergence is associated with a change in immune response from a predominant Th1 immune response to a Th2 immune response against tumour antigens.

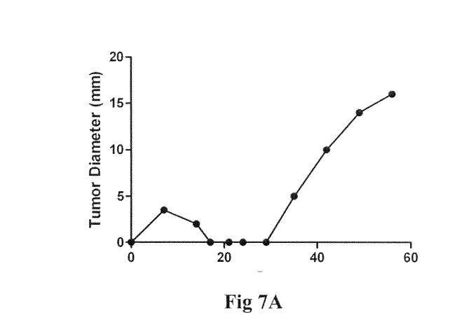

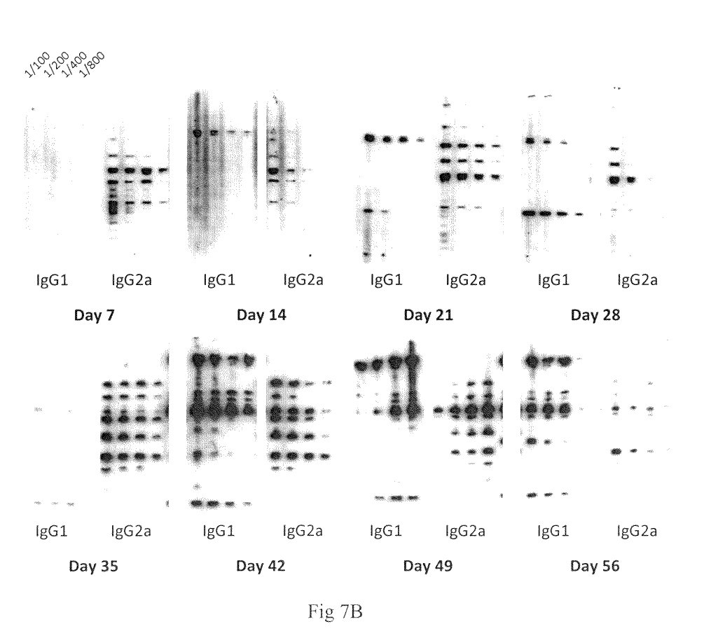

The mouse was implanted with 10⁵ P815 cells intradermally on day 0. The P815 tumour spontaneously resolved, and subsequently reappeared and grew progressively at the site of primary injection as is shown in FIG. 7A. The mouse was bled over a period of 10 weeks, and the relative predominance of P815-specific IgG1 and IgG2a antibodies were assessed by Western blot, illustrated in FIG. 7B. Four phases of the anti-tumour antibody response were noted. The first antibody was detectable at day 7, and was predominantly IgG2a reflecting a predominant Th1, cell-mediated immune response. The immune response evolved between days 14 and 28 to a mixture of IgG2a/IgG1 antibody. This time interval included a first phase of tumour growth, peak tumour formation, and tumour regression. About day 35, there was a subsequent change in the immune response from the previous predominant expression of IgG2a where the tumour was not evident, to tumour re-emergence and progression which involved a change in the immune response to the predominant expression of IgG1.

FIG. 7 clearly demonstrates the utility of the longitudinal measurement of the immune response in order to follow the changing nature of the anti-tumour immune response.

### Example 6

### Low-Dose Priming and the Effect on Immune Response

On generation of a primary immune response, the reduction of the antigen load by an appropriate amount may result in the modulation of an ongoing, mixed Th1/Th2 immune response to one with a predominant Th1 response.

Mice with foreign red blood cells (RBC) were immunized in a manner that resulted in the production of an antibody response. Analysis of the antibody produced indicated the generation of a mixed Th1/Th2 type immune response. The immune cells were harvested from the mice and were cultured to determine whether, and under what conditions, cells expressing CMI in the form of delayed-type hypersensitivity but no longer producing antibody could be generated. In the case where antigen was added to the culture, cells expressing CMI were generated. Cells cultured in the absence of antigen, the antigen-specific cells died.

These results indicate that lowering the amount of antigen present enables the modulation of the immune response from a mixed Th1/Th2 to a predominant Th1 response. However, lowering the amount of antigen too low results in the disappearance of immune response against the antigen.

In order to optimize chemotherapies during cancer treatment, continual assessment of the quantity and, in turn, type of immune response generated to the tumour is critical in order to establish an IgG isotype profile of the anti-tumour antibody. For example, in the case where the quantity of anti-tumour antibody is diminishing, treatment with low amounts of antigen may affect a shift toward a predominant Th1 response. Where the IgG isotype profile indicates a mixed Th1/Th2 response, treatment to modulate the response to a Th1 type, for example administering antibody that neutralizes IL-4, may be administered.

Further, the removal of a cancerous tumour by surgery or local radiation may enable the dramatic reduction in tumour load, and in turn, modulate the immune response to predominantly Th1.

### Example 7

### Decrease in CD4 T-Cells and the Effect on Immune Response

Mixed Th1/Th2 responses in mice may be modulated to a Th1 response by the partial depletion of their CD4 T cells. However, in order to be effective in cancer treatment this depletion should be established at level specific to a particular subject, as total depletion of the CD4 T-cells would obviate most CTL responses as the generation of most CTL responses is CD4 T-cell dependent and insufficient depletion would not enable immune response modulation.

Similar to Example 6, to optimize the cancer treatment, continual assessment of the quantity and type of immune response generated to the tumour is critical in order to establish an IgG isotype profile of the anti-tumour antibody.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The invention claimed is:

1. A method for modulating a type of immune response to a cancer treatment within a subject, said method comprising the steps of:
a) obtaining a sample from said subject;
b) assessing said sample to detect at least one IgG antibody or IgG isotype in said sample;
c) if said least one IgG antibody or IgG isotype is detected, identifying and quantifying said IgG antibody or IgG isotype expression;
d) comparing said quantified IgG antibody or IgG isotype with a suitable control, and determining changes in expression therebetween; wherein the changes in expression are indicative of the type of immune response to said cancer treatment;
e) characterizing said type of immune response;
f) repeating steps (a) through (e) for each of a series of spaced-apart time points;
g) assessing the series of characterized immune responses to determine the subject's responsiveness to the cancer treatment,
and modulating said cancer treatment to affect a change in the expression of said quantified IgG antibody or IgG isotype.

2. A method according to claim 1, wherein said sample is selected from the group consisting of cells, tissues, and fluids.

3. A method according to claim 2, wherein said cells are selected from the group consisting of cancerous cells, non-cancerous cells, and tumour cells.

4. A method according to claim 2, wherein said fluids are selected from the group consisting of blood and plasma.

5. A method according to claim 1, wherein said suitable control is selected from the group consisting of: a clinically selected control, a stored dataset of results generated from studies of the presence and expression of IgG antibody and IgG isotypes in one or more population(s) of subjects responsive to cancer treatment, a stored dataset of results generated from studies of the presence and expression of IgG antibody and IgG isotypes in one or more population(s) of subjects non-responsive to cancer treatment, a stored dataset of results generated from studies of the presence and expression of IgG antibody and IgG isotypes in samples collected from a single subject responsive to cancer treatment over an extended period of time, a stored dataset of results generated from studies of the presence and expression of IgG antibody and IgG isotypes in samples collected from a single subject non-responsive to cancer treatment over an extended period of time, and combinations thereof.

6. A method according to claim 5, wherein said clinically selected control is a physiological specimen responsive to said cancer treatment.

7. A method according to claim 5, wherein said clinically selected control is a physiological specimen non-responsive to cancer treatment.

8. A method according to claim 1, wherein said change in expression is an increase in said quantified antibody compared to said control and indicates an increased responsiveness to the cancer treatment.

9. A method according to claim 1, wherein said change in expression is a decrease in said quantified antibody compared to said control and indicates an increased responsiveness to the cancer treatment.

10. A method according to claim 1, wherein said change in expression is a decrease in said quantified antibody compared to said control and indicates a decreased responsiveness to the cancer treatment.

11. A method according to claim 1, wherein said change in expression is an increase in said quantified antibody compared to said control and indicates a decreased responsiveness to the cancer treatment.

12. A method according to claim 1, wherein said cancer treatment is selected from the group consisting of: surgery, chemotherapy, radiation therapy, immunotherapy, monoclonal antibody therapy, and combinations thereof.

13. A method for modulating a type of immune response to a cancer treatment within a subject, said method comprising the steps of:
a) obtaining a sample from said subject;
b) assessing said sample to detect IgG1 antibody in said sample;
c) if said IgG1 antibody is detected, quantifying said IgG1 antibody expression;
d) assessing said sample to detect IgG2a antibody in said sample;
e) if said IgG2a antibody is detected, quantifying said IgG2a antibody expression;
f) comparing said quantified IgG1 antibody and quantified IgG2a antibody with a suitable control, and determining changes in expression therebetween; wherein the changes in expression are indicative of the type of immune response to said cancer treatment;
g) characterizing said type of immune response;
h) repeating steps (a) through (g) for each of a series of spaced-apart time points;
i) assessing the series of characterized immune responses to determine the subject's responsiveness to the cancer treatment;
and modulating the cancer treatment to affect a change in the expression of said quantified IgG1 antibody and IgG2a antibody.

14. A method according to claim 13, wherein said sample is selected from the group consisting of cells, tissues, and fluids.

15. A method according to claim 14, wherein said cells are selected from the group consisting of cancerous cells, non-cancerous cells, and tumour cells.

16. A method according to claim 14, wherein said fluids are selected from the group consisting of blood and plasma.

17. A method according to claim 13, wherein said suitable control is selected from the group consisting of a clinically selected control, a stored dataset of results generated from studies of the presence and expression of IgG1 and IgG2a antibody in one or more population(s) of subjects responsive to cancer treatment, a stored dataset of results generated from studies of the presence and expression of IgG1 and IgG2a antibody in one or more population(s) of subjects non-responsive to cancer treatment, a stored dataset of results generated from studies of the presence and expression of IgG1 and IgG2a antibody in samples collected from a single subject responsive to cancer treatment over an extended period of time, a stored dataset of results generated from studies of the presence and expression of IgG1 and IgG2a antibody in samples collected from a single subject non-responsive to cancer treatment over an extended period of time, and combinations thereof.

18. A method according to claim 17, wherein said clinically selected control is a physiological specimen responsive to said cancer treatment.

19. A method according to claim 17, wherein said clinically selected control is a physiological specimen non-responsive to cancer treatment.

20. A method according to claim 13, wherein said change in expression is an increase in said quantified IgG2a antibody compared to said control and indicates an increased responsiveness to the cancer treatment.

21. A method according to claim 13, wherein said change in expression is a decrease in said quantified IgG1 antibody compared to said control and indicates an increased responsiveness to the cancer treatment.

22. A method according to claim 13, wherein said change in expression is a decrease in said quantified IgG2a antibody compared to said control and indicates a decreased responsiveness to the cancer treatment.

23. A method according to claim 13, wherein said change in expression is an increase in said quantified IgG1 antibody compared to said control and indicates a decreased responsiveness to the cancer treatment.

24. A method according to claim 13, wherein said cancer treatment is selected from the group consisting of surgery, chemotherapy, radiation therapy, immunotherapy, monoclonal antibody therapy, and combinations thereof.

25. A method of characterizing a type of immune response to a cancer treatment in a subject, said method comprising the steps of:
obtaining a sample from said subject;
assessing said sample to detect IgG1 antibody in said sample;
if said IgG1 antibody is detected, quantifying said antibody expression;
assessing said sample to detect IgG2a antibody in said sample;
if said IgG2a antibody is detected, quantifying said antibody expression;
comparing said quantified IgG1 and IgG2a antibody, and determining differences in the magnitude the expression therebetween;
wherein said differences in the expression of said quantified antibody are indicative of the type of immune response to said cancer treatment.

26. A method according to claim 25, wherein said sample is selected from the group consisting of cells, tissues, and fluids.

27. A method according to claim 26, wherein said cells are selected from the group consisting of cancerous cells, non-cancerous cells, and tumour cells.

28. A method according to claim 26, wherein said fluids are selected from the group consisting of blood and plasma.

29. A method according to claim 25, wherein said cancer treatment is selected from the group consisting of surgery, chemotherapy, radiation therapy, immunotherapy, monoclonal antibody therapy, and combinations thereof.

30. A method of characterizing a type of immune response to a cancer treatment in a subject, said method comprising the steps of:
obtaining a sample from said subject;
assessing said sample to detect IgG1 antibody in said sample;
if said IgG1 antibody is detected, quantifying said IgG1 antibody expression;
assessing said sample to detect IgG2a antibody in said sample;
if said IgG2a antibody is detected, quantifying said IgG2a antibody expression;
quantifying the ratio of expression of said quantified IgG2a antibody to IgG1 antibody;
wherein said ratio of expression of said quantified IgG2a antibody to IgG1 antibody is indicative of the type of immune response to said cancer treatment.

31. A method according to claim 30, wherein said sample is selected from the group consisting of cells, tissues, and fluids.

32. A method according to claim 31, wherein said cells are selected from the group consisting of cancerous cells, non-cancerous cells, and tumour cells.

33. A method according to claim 31, wherein said fluids are selected from the group consisting of blood and plasma.

34. A method according to claim 30, wherein said cancer treatment is selected from the group consisting of surgery, chemotherapy, radiation therapy, immunotherapy, monoclonal antibody therapy, and combinations thereof.

35. A method of modulating the type of immune response to a cancer treatment in a subject, said method comprising the steps of:
a) obtaining a sample from said subject;
b) assessing said sample to detect IgG1 antibody in said sample;
c) if said IgG1 antibody is detected, quantifying said antibody expression;
d) assessing said sample to detect IgG2a antibody in said sample;
e) if said IgG2a antibody is detected, quantifying said antibody expression;
f) quantifying the ratio of expression of said quantified IgG2a antibody to IgG1 antibody;
g) repeating steps (a) through (f) for each of a series of spaced-apart time points;
h) comparing the differences therebetween of the ratio of expression of the sample at each of said series of spaced-apart time points;
i) characterizing the type of immune response;
j) assessing the series of characterized immune responses to determine the subject's responsiveness to the cancer treatment,
and modulating the cancer treatment to affect a change in the ratio of expression of said quantified IgG2a antibody to IgG1 antibody.

36. A method according to claim 35, wherein said sample is selected from the group consisting of cells, tissues, and fluids.

37. A method according to claim 36, wherein said cells are selected from the group consisting of cancerous cells, non-cancerous cells, and tumour cells.

38. A method according to claim 36, wherein said fluids are selected from the group consisting of blood and plasma.

39. A method according to claim 35, wherein said cancer treatment is selected from the group consisting of surgery, chemotherapy, radiation therapy, immunotherapy, monoclonal antibody therapy, and combinations thereof.

40. A method according to claim 35, wherein said change in the ratio of expression of said sample is an increase, and indicates an increased responsiveness to the cancer treatment.

41. A method according to claim 35, wherein said change in the ratio of expression of said sample is a decrease, and indicates a decreased responsiveness to the cancer treatment.

42. A method of modulating a type of immune response to a cancer treatment within a subject, said method comprising the steps of:
a) obtaining a sample from said subject;
b) assessing said sample to detect IgG1 antibody in said sample;
c) if said IgG1 antibody is detected, quantifying said antibody expression;
d) assessing said sample to detect IgG2a antibody in said sample;
e) if said IgG2a antibody is detected, quantifying said antibody expression;
f) quantifying the ratio of expression of said quantified IgG2a antibody to IgG1 antibody;
g) quantifying the ratio of expression of a suitable control;
h) comparing said quantified ratio of expression of the sample to the ratio of expression of said control, and determining changes in the ratio of expressions therebetween, wherein the changes in said ratio of expression are indicative of the type of immune response to said cancer treatment;
i) characterizing said type of immune response;
j) repeating steps (a) through (i) for each of a series of spaced-apart time points;
k) assessing the series of characterized immune responses to determine the subject's responsiveness to the cancer treatment;
and modulating the cancer treatment to affect a change in the ratio of expression of said quantified IgG2a antibody to IgG1 antibody.

43. A method according to claim 42, wherein said sample is selected from the group consisting of cells, tissues, and fluids.

44. A method according to claim 43, wherein said cells are selected from the group consisting of cancerous cells, non-cancerous cells, and tumour cells.

45. A method according to claim 43, wherein said fluids are selected from the group consisting of blood and plasma.

46. A method according to claim 42, wherein said cancer treatment is selected from the group consisting of surgery, chemotherapy, radiation therapy, immunotherapy, monoclonal antibody therapy, and combinations thereof.

47. A method according to claim 42, wherein said suitable control is selected from the group consisting of a clinically selected control, a stored dataset of results generated from studies of the presence and expression of IgG antibody and IgG isotypes in one or more population(s) of subjects responsive to cancer treatment, a stored dataset of results generated from studies of the presence and expression of IgG antibody and IgG isotypes in one or more population(s) of subjects non-responsive to cancer treatment, a stored dataset of results generated from studies of the presence and expression of IgG antibody and IgG isotypes in samples collected from a single subject responsive to cancer treatment over an extended period of time, a stored dataset of results generated from studies of the presence and expression of IgG antibody and IgG isotypes in samples collected from a single subject non-responsive to cancer treatment over an extended period of time, and combinations thereof.

48. A method according to claim 47, wherein said clinically selected control is a physiological specimen responsive to said cancer treatment.

49. A method according to claim 47, wherein said clinically selected control is a physiological specimen non-responsive to cancer treatment.

50. A method according to claim 42, wherein said change in the ratio of expression of said sample compared to said control is an increase, and indicates an increased responsiveness to the cancer treatment.

51. A method according to claim 42, wherein said change in the ratio of expression of said sample to said control is a decrease, and indicates a decreased responsiveness to the cancer treatment.

52. A method according to claim 42, wherein said cancer treatment is selected from the group consisting of surgery, chemotherapy, radiation therapy, immunotherapy, monoclonal antibody therapy, and combinations thereof.

53. A method for modulating a type of immune response to a cancer treatment within a subject, said method comprising the steps of:
a) obtaining a sample from said subject;
b) assessing said sample to detect IgG1 antibody in said sample;
c) if said IgG1 antibody is detected, quantifying said IgG1 antibody expression;
d) assessing said sample to detect IgG2a antibody in said sample;
e) if said IgG2a antibody is detected, quantifying said IgG2a antibody expression;
f) comparing said quantified IgG1 and IgG2a antibody, and determining the difference therebetween;
g) characterizing said type of immune response;
h) repeating steps (a) through (g) for each of a series of spaced-apart time points;
i) assessing the series of characterized immune responses to determine the subject's responsiveness to the cancer treatment;
and modulating the cancer treatment to affect a change in the expression of said quantified IgG1 antibody and IgG2a antibody.

54. A method according to claim 53, wherein said sample is selected from the group consisting of cells, tissues, and fluids.

55. A method according to claim 54, wherein said cells are selected from the group consisting of cancerous cells, non-cancerous cells, and tumour cells.

56. A method according to claim 54, wherein said fluids are selected from the group consisting of blood and plasma.

57. A method according to claim 53, wherein said change in expression is an increase in said quantified IgG2a antibody, and indicates an increased responsiveness to the cancer treatment.

58. A method according to claim 53, wherein said change in expression is a decrease in said quantified IgG1 antibody compared, and indicates an increased responsiveness to the cancer treatment.

59. A method according to claim 53, wherein said change in expression is a decrease in said quantified IgG2a antibody compared, and indicates a decreased responsiveness to the cancer treatment.

60. A method according to claim 53, wherein said change in expression is an increase in said quantified IgG1 antibody, and indicates a decreased responsiveness to the cancer treatment.

61. A method according to claim 53, wherein said cancer treatment is selected from the group consisting of: surgery, chemotherapy, radiation therapy, immunotherapy, monoclonal antibody therapy, and combinations thereof.

62. A method of quantifying a type of immune response to a cancer treatment within a subject, said method comprising the steps of:
a) obtaining at least two samples from said subject;
b) harvesting cells from said samples;
c) exposing said cells from one of said samples to an anti-IgG1 antibody;
d) exposing the cells from the second of said samples to an anti-IgG2a antibody;
d) incubating the cells from said samples;
e) washing the cells from said samples with a buffer solution;
f) quantifying the expression of IgG1 and IgG2a antibody in said first and second samples;
wherein the quantified antibody is indicative of the magnitude of the type of immune response to the cancer treatment.

63. A method according to claim 62, wherein said sample is selected from the group consisting of cells, tissues, and fluids.

64. A method according to claim 63, wherein said cells are selected from the group consisting of cancerous cells, non-cancerous cells, and tumour cells.

65. A method according to claim 63, wherein said fluids are selected from the group consisting of blood and plasma.

66. A method according to claim 62, wherein said cancer treatment is selected from the group consisting of surgery, chemotherapy, radiation therapy, immunotherapy, monoclonal antibody therapy, and combinations thereof.
